(19) United States
(12) Plant Patent Application Publication
(10) Pub. No.: US 2013/0227751 P1
(43) Pub. Date: Aug. 29, 2013

(54) Orange tree named 'KARNINKA'

(50) ;

(71) Applicant: Daniel Frederich Rautenbach, Patensie (ZA)

(72) Inventor: Daniel Frederich Rautenbach, Patensie (ZA)

(73) Assignee: Citrogold (Pty) Ltd, Stellenbosch (ZA)

(21) Appl. No.: 13/385,675

(22) Filed: Feb. 27, 2012

(51) Int. Cl. A01H 5/00

(52) U.S. Cl. PLT/202

(57) ABSTRACT

A new and distinct variety of sweet orange tree (Citrus sinensis) named ‘Karninka.’ ‘Karninka’ is notable for its firm, late maturing fruit.

### CROSS-REFERENCE TO RELATED APPLICATIONS

None

### GENUS AND SPECIES

Citrus sinensis

### VARIETY DENOMINATION

‘Karninka’

### BACKGROUND AND SUMMARY OF THE VARIETY

The new orange tree ‘Karninka’ originated as a whole tree mutation of a ‘Palmer’ navel orange tree (not patented). The mutation was discovered in 2005 in a commercial orchard of ‘Palmer Navel’ trees on ‘Swingle’ citrumelo rootstock (not patented) at Ripplehill Farm, Patensie, South Africa, and was distinguished by its markedly late maturity as compared to other navel orange trees, even those considered to be late-maturing. Asexual propagation by budding was first carried out in 2005 at Patensie to determine whether the desirable characteristics of the mutation would carry through to asexually propagated progeny. It has been found that the desirable characteristics are in fact reproduced through asexual propagation, and remain stable through successive generations.

‘Karninka’ is a new and distinct orange tree notable for its late maturity as compared to ‘Palmer Navel’ and other late maturing orange varieties.

### BRIEF DESCRIPTION OF THE PHOTOGRAPHS

FIG. 1 shows the fruit of the new variety;

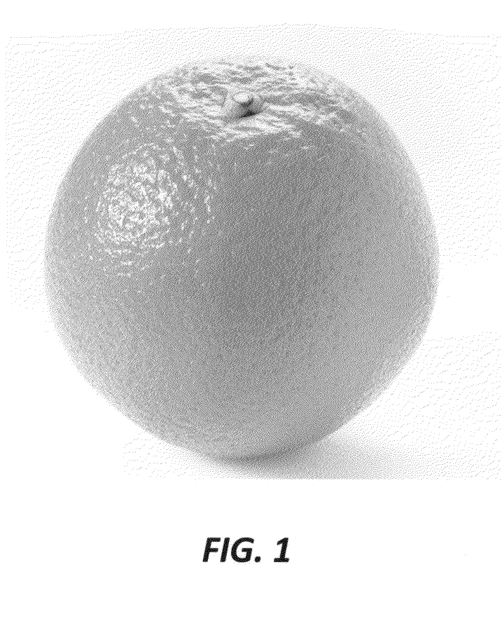

FIG. 2 shows a sectioned fruit of the variety;

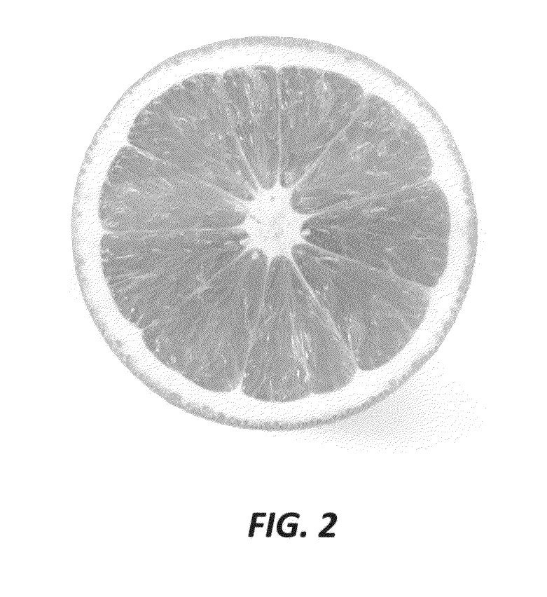

FIG. 3 shows fruit segments of the new variety; and

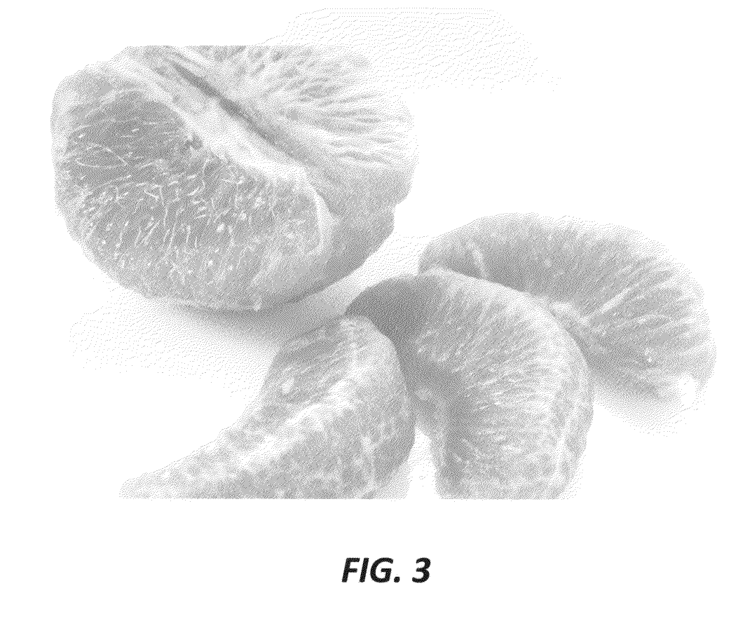

FIG. 4 shows the tree and leaves of the new variety.

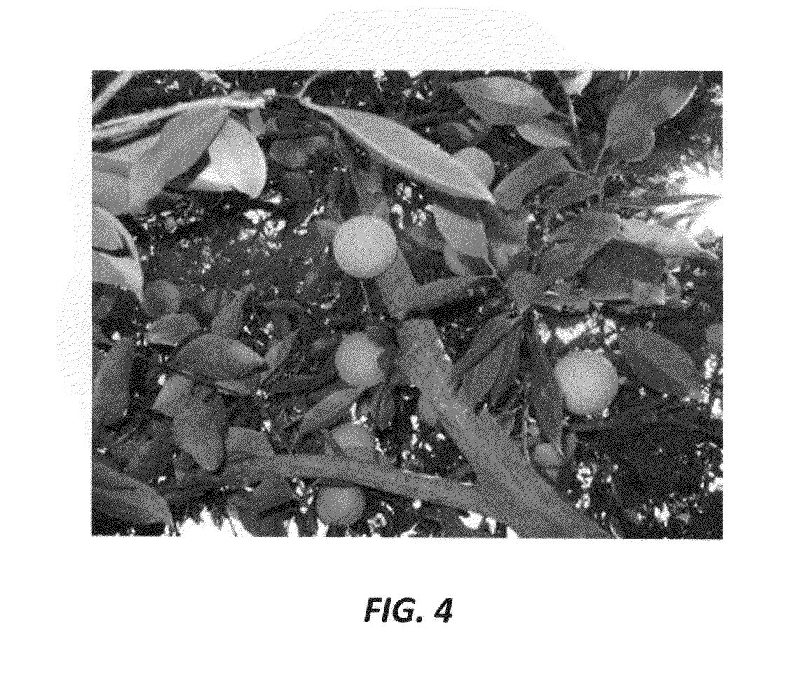

The colors of the claimed variety may vary with lighting conditions. Color characteristics of the variety should therefore be determined with reference to the observations described herein, rather than from these illustrations alone.

### DETAILED BOTANICAL DESCRIPTION

The following detailed botanical description is based on observations of the original tree of the variety, planted in 1983 and grown on ‘Swingle’ citrumelo rootstock. Observations were recorded and photographs taken during the 2008, 2009 and 2010 growing seasons at Patensie, South Africa. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants of the new variety may vary from the stated average. Colors are described with reference to The Royal Horticultural Society Colour Chart (2007) (RHS) and the Citrus Research International Colour Prints (Nelspruit, South Africa, 2005).
Tree:
   Vigor.—Medium, comparable to ‘Palmer’ navel.
   Density of canopy.—Average to dense.
   Habit.—Spreading.
   Height.—1.6 m, similar to ‘Palmer’ navel.
   Spread.—Medium, 1.3 m.
   Trunk diameter.—17 mm (one of two stems, 30 cm above graft).
   Bark texture.—Reasonably smooth, typical of navel orange trees.
   Bark color.—Dark grey with green striae, typical of navel orange trees.
   Tendency toward alternate bearing.—None noted.
   Winter hardiness.—Similar to ‘Washington’ navel.
   Chilling requirement.—Similar to ‘Washington’ navel.
   Drought tolerance.—Similar to ‘Washington’ navel.
Branches:
   Length.—30 cm.
   Diameter.—5 mm.
   Crotch angle.—45° maximum.
   Bark color.—Green.
   Bark texture.—Smooth.
   Thorns present?.—Yes.
   Thorn length.—Short, up to about 8 mm.
   Current year shoot length.—Up to 250 mm.
   Current year shoot color.—Light green.
Flowers:
   Quantity of blossoms per cluster.—Up to 4.
   Blossom diameter.—49.5 mm.
   Blossom depth.—27.0 mm (base of sepal to apex of petal).
   Pollen present?.—Yes.
   Sepal length.—5.71 mm.
   Sepal width.—4.29 mm.
   Sepal shape.—Acute.
   Sepal margin.—Entire (smooth).
   Sepal color — upper surface.—Very light green with white margins.
   Sepal color — lower surface.—Light green with white margins.
   Quantity of petals per flower.—Generally 5.
   Petal shape.—Acute, elongated, stellate.
   Petal apex.—Acute.
   Petal margin.—Entire.
   Petal length.—25.92 mm.
   Petal width.—744 mm.
   Petal color — upper surface.—White.
   Petal color — lower surface.—White with yellow dots.
   Date of first bloom.—10 October.
   Date of full bloom.—15 October.
   Date of first fruitlet fall.—14 November.
   Pedicel length.—10.66 mm.
   Pedicel diameter.—1.66 mm.
   Pedicel color.—Light green.
   Pistil quantity per flower.—1.
   Pistil length.—15.61 mm.
   Pistil color.—Green, light green, pale yellow.
   Anther quantity per flower.—25.
   Anther length.—1.57 mm.
   Anther color.—Yellow brown.
   Stigma quantity per flower.—1.
   Stigma length.—2.36 mm.
   Stigma color.—Yellow.
   Style quantity per flower.—1.
   Style length.—10.09 mm.
   Style color.—Very light green.
   Ovary quantity per flower.—1.
   Ovary length.—4.38 mm.
   Ovary diameter.—4.43 mm.
   Ovary color.—Light green.
Leaves:
   Length.—120 to 130 mm.
   Width.—65 to 70 mm.
   Length to width ratio.—1:1.77.
   Margin.—Entire to very slightly undulate.
   Color.—Upper surface — Green 143A; Lower surface Green 142B.
   Texture.—Glabrous.
   Leaf shape.—Elliptic.
   Apex shape.—Acuminate.
   Base shape.—Equilateral, cuneate.
   Petiole length.—20.1 mm.
   Petiole diameter.—3 mm.
   Petiole color.—Light green to green.
   Wings.—Present.
   Wing width.—12 mm.
Fruit:
   Axial diameter.—81 to 85 mm.
   Apical diameter.—79 mm.
   General shape in profile.—Round.
   Navel.—Absent to small — 30% 6 to 8 mm.
   Rind color.—Yellow-orange 21B; compares to color plate 1 of set no. 34 for oranges, where plate 1 is full colored orange and plate 8 is green (Colour Prints for Blemish Standards, Citrus Research Council).
   Oil glands per cm².—33.
   Oil gland diameter.—0.8 to 0.9 mm.
   Rind thickness.—Thin, 5 to 9 mm.
   Ease of peeling.—Moderate.
   Rind texture.—Medium.
   Albedo.—White.
   Quantity of segments per fruit.—9 to 11.
   Toughness of segment membrane.—Tough initially, becoming soft.
   Juice sac length.—Medium, about 11 to 13 mm.
   Juice sac shape.—Elongated.
   Juice sac length to width ratio.—1:4.
   Juice sac color.—Almost transparent to faint orange.
   Juice soluble solids (° Brix): 13° to 15°.
   Seeds present?.—No.
   Relative harvest maturity.—Late.
   Harvest window.—September.
   Market use.—Late harvest fresh.

TABLE 1
Fruit internal quality tests

| | | Fruit | | | | | |
|---|---|---|---|---|---|---|---|
| | | Size | Juice | | Acid | | Average |
| Date | Color | (mm) | % | TSS | % | Ratio | Seed |
| 23 Jul., 2008 | 1-3 | 86-89 | 50.2 | 14.0 | 1.53 | 9.2 | 0 |
| 8 Aug., 2008 | 1 | 81-85 | 55.9 | 14.0 | 1.62 | 8.6 | 0 |
| 21 Aug., 2008 | 1 | 81-85 | 52.3 | 14.7 | 1.48 | 9.9 | 0 |
| 4 Sep., 2008 | 1 | 81-85 | 54.2 | 14.9 | 1.48 | 10.0 | 0 |
| 17 Sep., 2008 | 1 | 81-85 | 54.7 | 14.9 | 1.34 | 11.1 | 0 |
| 16 Jul., 2009 | 1 | 81-85 | 55.1 | 13.1 | 1.29 | 10.2 | 0 |
| 30 Jul., 2009 | 1 | 77-80 | 57.5 | 13.7 | 1.28 | 10.7 | 0 |
| 6 Aug., 2009 | 1 | 77-80 | 51.7 | 13.5 | 1.23 | 11.0 | 0 |
| 17 Aug., 2009 | 1 | 81-85 | 58.4 | 13.4 | 1.21 | 11.1 | 0 |
| 24 Aug., 2009 | 1 | 77-80 | 58.3 | 13.9 | 1.30 | 10.7 | 0 |
| 2 Sep., 2009 | 1 | 77-80 | 59.3 | 14.0 | 1.24 | 11.3 | 0 |
| .10 Sep., 2009 | 1 | 81-85 | 52.0 | 14.8 | 1.03 | 14.4 | 0 |

Color rating: Color transparency rating measured from 1 to 8: 1 = Well colored mature fruit with no green evident; 8 = Green fruit with no signs of color break.
Internal quality tests of fruit of the ‘Karninka’ navel tree. Fruit from the parent tree, Ripplehill, Patensie on ‘Swingle’ citrumelo rootstock

1. A new and distinct variety of orange tree, substantially as illustrated and described herein
